(19) United States
(12) Patent Application Publication
(10) Pub. No.: US 2024/0043468 A1
(43) Pub. Date: Feb. 8, 2024

(54) POLYMER FOR PURIFICATION OF BIOMOLECULES

(71) Applicant: PURIUS LTD, London (GB)

(72) Inventor: Michael Harry ROSE, London (GB)

(21) Appl. No.: 18/254,104

(22) PCT Filed: Nov. 23, 2021
(86) PCT No.: PCT/GB2021/053021

§ 371 Date: May 23, 2023

(30) Foreign Application Priority Data
Nov. 23, 2020 (GB) 2018381.0

(51) Int. Cl. C07K 1/22; C07K 16/06

(52) U.S. Cl. CPC C07K 1/22; C07K 16/065; C07K 2317/515; C07K 2319/00; C07K 2317/22

(57) ABSTRACT

There is disclosed a composition comprising a first construct, wherein the first construct comprises a charge component, a lock component and a key component; wherein the lock component and the key component are specific affinity binding partners and are separated such that they are unable to form a binding pair within the same construct; whereby in a first environmental condition, the charge component imparts a first overall charge to the first construct, the lock component binds to its binding partner on a second construct, and the key component binds to its binding partner on a third construct; and whereby in a second environmental condition, the charge component imparts a second overall charge to the first construct, and the lock and key components are in an unbound state. Also disclosed is a method of purification of a target compound involving the composition.

### FIELD OF THE INVENTION

The present invention relates to the selected and specific purification of target complex compounds from a liquid solution mixture containing impurities via a smart ‘construct’ designed to alternately polymerise and monomerise in reaction to changes in the solution environment.

### BACKGROUND TO THE INVENTION

Purification of complex compounds is a high value industrial processing step typically involving expensive and time intensive operations. Often these compounds are products or product intermediates used for medical applications and depend on a highly specific molecular structure to perform their function and efficacy.

In many cases and for many medical applications these compounds come in the form of biomolecules and are synthesised in a biological process via genetic engineering of a host cell line grown in a large cell culture in a liquid suspension in a bioreactor and the cell line is often genetically engineered to make a specific variant of the target molecule and in large quantities. An industrial challenge in these bio production processes is that a large assortment of by products are also made in the cell and in large quantities, and these by products, typically including functional macromolecules, reaction intermediates and substrates, and large cell structures, are often deleterious and toxic for the working application of the target molecule such as in a medical drug to be applied within human body. Therefore, it is common practice to selectively remove these impurities to as great an extent as possible. Furthermore, the target product and the impurities are often suspended in a solution environment that is not conducive to the long term stability of the product (or the impurities) and must be re-dissolved in a solution that is safe and appropriate for the application. The quantity and concentration of the product is also often much lower than required in the final formulation of the product for its purpose (often medical). In industrial manufacturing processes, purifying and re-suspending the product is often achieved through a combination of selection steps such as chromatography and membrane filtration.

For high specificity of a purification step a common practice called chromatography is to have a ligand that reversibly binds preferentially to the target product compared to the impurities or vice versa and to have such a ligand immobilised in place, typically on a material with a very high surface area and immersed in the volume of the impure suspension. The immobilisation of such a ligand allows binding of the product while the impurities flow past and are displaced by a washing fluid such as a buffered solution, or vice versa where the impurities are captured by the ligand leaving the product free in the suspension. Such ligands are usually chemically immobilised on a substrate with high porosity and surface area in order to maximise mass transfer rates, and such substrates are often formed into microscopic porous beads in order to pack them into a column capped on each end with mesh frits allowing the pumping of fluids including the product containing impure mixture and other fluids through the column and past the ligands immobilised on the substrate. Afterwards in a process called elution a change in the washing solution environment can be used to cancel or reverse the binding propensities of the ligand so that the bound component is released and the system can be reset to be used once again. If the target product was the bound component and depending on how the elution buffer is distributed then during this elution step the product may be released into a smaller volume and necessarily a different solution environment, allowing a potential increase in concentration and an exchange into a differing solution environment. A very high level of purification can be achieved by increasing the binding specificity of the ligand for its counterpart. A ligand designed to specifically bind only the target product molecule through precisely complimentary structure and charge distributions can be said to be affine or of an affinity type and such a chromatography is known as an affinity chromatography. Commonly in industrial practice such an affinity ligand is itself a complex molecule and must itself be manufactured in a complex biological purification process, making it of comparable expense to the target product itself. Another type of ligand may carry a strong positive or negative charge and through a chromatography process known as ion exchange chromatography the product target produce can be selected through attraction or rejection as the target molecule is typically charged. Often the target molecule contains a combination of acidic and basic sub-residues and depending on the pH of the solution environment the relative level of dissociation of these residues leaves the product with different charge levels at different pHs, and at a balancing pH known as the pI it may leave the target molecule at an overall neutral charge. By modulating the pH and by selecting the appropriately charged ligand for the chromatography step, the target molecule can be attracted to the ligand if they have opposite charges or rejected from the ligand if they have like charges, and since other impurity molecules in the fluid suspension may have different charge properties, in the same suspension these may be complimentary rejected from the ligands or bound, resulting in a level of purification similar to the mechanism described for affinity separations. In the mode where the target molecule binds to the ligand and is then separately removed from it again via an elution buffer, impurities possessing a similar charge may also co-bind to the ligand, but those impurities often possess a different acid/base residue composition to the target molecule and possess different levels of charge at different pHs. During the elution step the product of interest may be released from the ligand by changing the environment pH thereby changing its charging level and therefore binding strength, or by introducing an excess of charged molecules such as dissolved salt ions, which preferentially bind the ligand and displace the product off, and since similarly bound impurities will often have different charge levels or differing charges levels at different pHs, these co-bound impurities may be more strongly or more weakly bound than the target product as the solution environment changes. By changing the solution environment gradually this causes the bound molecules to be released in sequence, allowing for separated release of the target product and the impurities thereby allowing for a finely tuned purification.

A separate step known as ultrafiltration/diafiltration may also be used to change the solution environment of the target product and to change its concentration and to perform purification. In this processing step a membrane is formed out of a porous substrate having a porosity of a controlled size distribution. The impure fluid suspension containing the target product may be pressed against one surface of the membrane called the retentate side under pressure and molecules smaller than the pore size of the membrane including the suspension fluid and macroscopic molecules such as proteins may squeeze through to the other side of the membrane called the permeate side, leaving the remaining molecules on the retentate side more concentrated. If the product is larger than the pore size then in this way it may be concentrated in the retentate. By adding another fluid to the retentate it can displace the fluid that was lost through the membrane to the permeate side and by doing a lot of this the suspension fluid can be exchanged with a new fluid to an extremely high degree. This process is very slow and is often sped up through mechanisms to decrease the local viscosity on the retentate side, such as by applying a strong crossflow' parallel to the membrane surface.

These are the primary tools in purification of complex macromolecules and in both instances require the mass transfer of components suspended or dissolved in a 3-dimensional bulk fluid against a 2-dimensional surface with complex surface properties and this is a fundamentally slow process. In order to speed this up in both instances the surfaces are highly engineered and the flow carefully controlled to maximise the speed of these exchanges, and both the surface properties (such as ligands or pore sizes) and the required engineering (such as microbead immobilisation or directional crossflow) lead to these materials being very expensive and their operation both complex and slow. In the field of biopharmaceutical manufacture these processing steps, often applied in multiple passes are responsible for the extreme expense of the cost of these drugs, which can be prohibitive for consumers.

### SUMMARY OF THE INVENTION

References to “a construct” refer to the “first construct”. Embodiments relating to the “first construct” equally apply to the “second construct” and the “third construct”. References to a “different construct” and “distinct construct” refer to another copy of the “first construct” or the “second construct” or the “third construct”. The first, second or third constructs are themselves specifically arranged polymers of components.

“Polymerisation” refers to the association of multiple constructs to form a chain or polymer. For example, multiple copies of the first construct may bind to each other to form a chain. The first construct may bind to the second construct which may bind to the third construct and so on to form a chain.

“Monomerisation” refers to the dissociation of a chain of polymerised constructs to individual monomeric construct units, the monomeric units being the unbound constructs, i.e. the first construct is a monomeric unit.

“Specific affinity binding partner” refers to two counterpart compounds that spontaneously bind to each other in the correct environmental conditions and whose binding is highly specific, largely rejecting binding of non-target compounds and whose specificity is typically enabled by their complex, specific and complementary structure. Alternate compounds may also possess a near identical complementary region and thus also specifically bind the counterpart. The specificity means that even small modifications to the binding site of either of the binding pairs will result in significant degradation or enhancement of binding propensity. Affinity binding is typically dependant on the collective attractive properties of many weaker complementary interactions, and as such are usually reversible, often via changes to the solution environment.

There is provided a composition comprising a first construct, wherein the first construct comprises:
(i) a charge component;
(ii) a lock component; and
(iii) a key component.

The lock component and the key component are specific affinity binding partners and are separated on the construct such that they are unable to form a binding pair within the same construct.

In a first environmental condition, the charge component imparts a first overall charge to the first construct, the lock component binds to its binding partner on a second construct, and the key component binds to its binding partner on a third construct.

In a second environmental condition, the charge component imparts a second overall charge to the first construct, and the lock and key components are in an unbound state.

The composition provides a mechanism of purification and resuspension of a target compound from a mixture of impurities suspended in a fluid environment. In particular, valuable biomolecules, such as monoclonal antibodies, may be purified from an aqueous solution comprising cellular debris and protein, nucleotide, carbohydrate and metabolic substrate intermediate by products from a biological culture. The first construct may be composed of several components in a chain, including both partnered components of a lock and key pair that are held apart. The lock and key components may form affinity bonds with their counterparts, possessing a high specificity that excludes non-specific binding, and whose binding properties may be such that they are reversible under changes to the solution environment, such as the pH. As the lock and key components on an individual construct may be held apart rigidly they have a low propensity for folding over and binding to each other within one copy of the construct. However, the lock component of the first construct may bind to the key component of another construct (i.e. the second construct), and many copies may therefore polymerise into a long chain of potentially unlimited length. By controlling the solution environment to modulate this binding, the construct may be induced to form a macroscopic polymer globule/gel, or may be induced to re-monomerise, and such a process may be repeated many times, and owing to the specificity of the bonding, predominantly copies of the construct may be taken up into the globular form when polymerising, leaving the majority of suspended impurities behind in the bulk volume. The bulk polymer may be sifted by a macroscopic coarse-grained filter, and redeposited in a second solution where it may be re-monomerised, in a new cleaner solution environment with different properties, and at a new concentration compared to the original solution environment.

The first construct may be any suitable polymer. For example, the construct may be a polypeptide, nucleic acid, lipid, organic polymer or synthetic polymer. In some embodiments, the construct comprises amino acids, nucleotides, or a combination thereof. In some embodiments, the construct consists essentially of amino acids. In some embodiments, the construct consists essentially of nucleotides.

Preferably, the polymer is a polypeptide. The charge component, lock component, key component, cleavage component and cleavage site may be composed of the same or different molecular types, e.g. peptide, polypeptide, nucleic acid, lipid, organic polymer or synthetic polymer. Preferably, the components are composed of the same molecular type, and more preferably, the components are composed of peptides and/or polypeptides.

In some embodiments, the first construct may be pH-responsive.

Each charge component, lock component and key component may be a globular tertiary structure. Each component may be largely unfolded, unfolded and/or linear. In some embodiments, some of the components are globular tertiary structures and some of the components are linear structures. Preferably, each component has a globular tertiary structure. More preferably, each component has a globular tertiary structure and the components are linked together in a linear chain.

Preferably, the lock and key components are directly encoded into the construct. Preferably, the lock, key and charge components are directly encoded into the construct. By “directly encoded”, it is meant that the code for the construct (e.g. the DNA or RNA code) includes the code for the lock and key components, such that a single construct code can be expressed to form a final construct that intrinsically includes the lock and key components. For example, a single oligonucleotide coding for the construct may be expressed to form a single polypeptide that includes the lock and key components. In a further example, a single oligonucleotide coding for the construct may be expressed to form a single polypeptide that includes the lock, key and charge components. In other examples, where the final version of the construct is a polynucleotide, a single oligonucleotide coding for the construct may intrinsically include the lock and key, or lock, key and charge components in the single oligonucleotide.

The components of the construct may be in any order. The following order is envisaged and is not limiting to the invention:
Key-lock-key-charge component

The target compound is produced separately and remains free in solution.

The components may be arranged in any order with components being swapped in and out for bespoke design. For polypeptide constructs, the N and C terminals may be reversed. Particular design rules may be used to enhance the functionality of the system:

The lock and key components may be adjacent to each other, i.e. directly next to each other or linked by a small and/or rigid linker. This design provides the advantage that the components do not have the flexibility to fold back and form a binding pair within the same construct.

Non-limiting examples of lock and key affinity binding pairs include:
Surface cell wall proteins (e.g. Protein A, Protein L, Protein G) which specifically bind antibodies (“antibodies” including monoclonal antibodies (mAbs), fragment antibodies (fAbs), Bi-specific T-cell engagers (BiTEs), affibodies, Camelids etc.);
Antigens pairing with antibodies raised against said antigens (“antibodies” including monoclonal antibodies (mAbs), fragment antibodies (fAbs), Bi-specific T-cell engagers (BiTEs), affibodies, Camelids etc.);
Hormones/signal transduction pathway proteins binding their specific receptor partners;
Interfering RNA (e.g. miRNA, siRNA) binding oligonucleotides;
DNA Repressor proteins binding oligonucleotides;
DNA Aptamers binding target protein ligands.

In some embodiments, the lock component may be an antibody, antibody fragment or antibody mimetic, and the key component may be a surface cell wall protein A, a surface cell wall protein L, or a surface cell wall protein G.

The charge component may be located anywhere in the construct. Preferably, the charge component is located at one end of the construct. This provides the advantage that the charge would be most exposed and potentially most potent. Where the charge component comprises histidine residues, the histidine residues may be located at one end of the polymer allowing dual functionality as a charge component and a histidine tag.

The constructs disclosed above and throughout the specification are particularly envisaged as polypeptide constructs. The constructs disclosed above and throughout the specification are particularly envisaged as polypeptide constructs that are pH responsive, i.e. the first, second and third environmental conditions are a first, second and third pH. The constructs disclosed above and throughout the specification are particularly envisaged as polypeptide constructs that are pH responsive and comprise rigid interlinks between the components. The constructs disclosed above and throughout the specification are particularly envisaged as polypeptide constructs that are pH responsive and comprise rigid interlinks between the components and further comprise a reversibly bound target compound. The constructs disclosed above and throughout the specification are particularly envisaged as constructs that are pH responsive and comprise rigid interlinks between the components. The constructs disclosed above and throughout the specification are particularly envisaged as constructs that are pH responsive and comprise rigid interlinks between the components and further comprise a reversibly bound target compound.

The second construct may be identical or substantially identical to the first construct. The second construct may include all of the same essential elements as the first construct but differ in the non-essential elements. Upon reading the appended claims, the skilled person would instantly recognise the essential elements of the first construct. Preferably, the second construct is identical or substantially identical to the first construct. More preferably, the second construct is identical to the first construct.

The second construct may comprise:
(i) a charge component;
(ii) a lock component; and
(iii) a key component.

The second construct may further comprise:
(i) additional key components;
(ii) additional lock components;
(iii) one or more target compounds; and/or
(iv) rigid interlinks.

The second construct may be pH-responsive.

The embodiments relating to the second construct equally apply to the third construct. Preferably, the third construct is identical or substantially identical to the first construct. Preferably, the third construct is identical or substantially identical to the second construct. More preferably, the third construct is identical to the first construct and the second construct.

The third construct may comprise:
(i) a charge component;
(ii) a lock component; and
(iii) a key component.

The third construct may further comprise:
(i) additional key components;
(ii) additional lock components;
(iii) one or more target compounds; and/or
(iv) rigid interlinks.

The third construct may be pH-responsive.

The charge component may be any component that assigns an overall charge to the construct wherein the overall charge differs in different environmental conditions, i.e. the construct has a first overall charge in one condition and a second overall charge in a second condition. In some embodiments, the environmental condition is selected from environmental pH, temperature, pressure, acoustic vibration, magnetic field density, electric field density, electromagnetic excitation, local component concentration, and the presence or absence of a co-enzyme or cofactor or similar activator compound. Preferably, the environmental condition is pH, i.e. the pH of a solution comprising the composition.

In some embodiments, the environmental condition is salt. The specific affinity binding pair may be selected or engineered to be sensitive to the environmental salt condition, by way of destabilising the affinity partner bond through destabilising of the constituent proteins, including their solubility in their environment. Salting environments could include Cationic partners such as Sodium, Potassium, Calcium, Nickel, Copper, Magnesium, Zinc, Iron, Magnesium, Lithium, guanidinium, ammonium and urea, and

Anionic partners can include Chlorine, Bromine, Phosphorus and phosphates, Sulphur and sulphates, acetate, citrate etc. Particular pairwise examples include Sodium Chloride, Sodium Phosphate, Sodium Acetate, Potassium Chloride and guanidinium chloride. For salting examples on the Hoffmeister series such as guanidinium chloride or Urea, the Protein A/Antibody affinity binding has been shown to be destabilised with increasing concentrations of said components as they destabilise the Protein A/Antibody structures. Simultaneously increasing salt concentrations will reduce relative solubility of the Protein A/Antibody components, also destabilising the binding such as in the case of increasing ammonium sulphate conditions.

In various embodiments, the environmental condition is temperature. For instance a complementary set of DNA strands may be used as the affinity binding pair and may be covalently bound to the other components in the construct. Alternatively, they may take advantage of DNA tertiary structure to specifically bind to other components of the construct such as in the mechanism of DNA-Histone binding. Both mechanisms provide the advantage that they are reversibly sensitive to the environment temperature, melting into separate strands at a high temperature and specifically reannealing at a low temperature. Different DNA constructs with different levels of complementarity, GC content, and lengths may be used to tune the sensitivity of the melting point to the environmental temperature.

In several embodiments, the environmental condition is a non-aqueous solvent. For instance, the affinity binding pair may be released in the increased presence of a non-aqueous solvent. The non-aqueous solvent may be, for example, ethanol, methanol, ethylene glycol, soluble polyethylene glycol, or dimethyl sulfoxide, which may be dissolved in an aqueous solution, or may displace a water solvent entirely leaving just the liquid solvent on its own.

The charge component has the advantage that it can be designed with a controlled, programmable charge. This allows for controlled inducement of charge of the overall polymer by controlling the environment condition, for example by controlling the pH. During the polymerisation process this is of particular use as having an excess of negative charge during the polymerisation can actively repel negatively charged impurities, further enhancing the polymerisation specificity. Performing the polymerisation in another pass but with a positive excess charge can likewise exclude positively charged components. Furthermore, careful control of overall charge may modulate self-repulsion of the construct, allowing for tuning of the structural packing of the polymer.

In some embodiments, the first environmental condition is a first pH and the second environmental condition is a second pH. The second pH may be more acidic compared to the first pH. The first pH may be between 5 to 9 and the second pH may be between 2 to 6. In some embodiments, the first pH may be between 6 to 8 and the second pH may be between 3 to 5. The second pH may be at least 1.5 pH units lower than the first pH. The first and second pH may be neutral or near-neutral.

In some embodiments, the charge component comprises a plurality of subunits that are charged. For example, the charge component may comprise a plurality of acidic and/or basic and/or zwitterionic chemically dissociating compounds. Preferably, the charge component is a polypeptide comprising charged residues. More preferably, the charge component is a polypeptide comprising a plurality of amino acids selected from aspartate, glutamate, lysine, arginine and histidine. Even more preferably, the charge component is a polypeptide comprising a plurality of amino acids with acidic side chains and a plurality of amino acids with basic side chains. Even more preferably, the charge component is a polypeptide comprising a plurality of histidine residues and a plurality of aspartate and/or glutamate residues.

If an acidic or basic or zwitterionic sub-unit is incorporated containing a pI that is close to neutral, then by controlling the environmental pH to control chemical dissociation the overall charge may be switched from charged to neutral, or the overall charge flipped from one charge to the other, or in the case of zwitterionic compounds, may be flipped individually from one charge to the other.

For example, the charge component may be a polypeptide and may comprise a number of acidic residues such as glutamate or aspartate that dissociate at neutral conditions to give a negative charge, and a large number of histidine residues that dissociate at neutral conditions to give a positive charge, thus giving the whole unit an overall positive charge. Since histidine has a pI slightly below neutral, reducing the environmental pH to slightly below this pI can induce a proportion of these histidine residues to re-associate, turning them neutral, and the number of remaining charged histidine residues may be balanced with the number of overall acidic residues giving an overall neutral charge to the charge component. If the pH is dropped further then more of the histidine residues can re-associate, leaving the charge component overall negatively charged. By controlling the excess number of histidine residues, the ratio of dissociation/re-association can be adjusted for a given pH giving programmable pH sensitivity. Thus, the charge component and in turn the construct and the polymer can have an overall charge that is programmable and controllable.

In various embodiments, the charge component is a polypeptide comprising at least 4 amino acid residues selected from histidine, aspartate and glutamate. In some embodiments, the charge component is a polypeptide comprising at least 5, 6, 7, 8, 9 or amino acid residues selected from histidine, aspartate and glutamate. In several embodiments, the charge component is a polypeptide comprising up to 5, 6, 7, 8, 9 or 10 amino acid residues selected from histidine, aspartate and glutamate.

In some embodiments, at least 10% of the residues of the charge component are negatively charged at pH 7.0. In some embodiments, at least 20% of the residues of the charge component are negatively charged at pH 7.0. In some embodiments, at least 30% of the residues of the charge component are negatively charged at pH 7.0.

In some embodiments, at least 10% of the residues of the charge component are positively charged at pH 7.0. In some embodiments, at least 20% of the residues of the charge component are positively charged at pH 7.0. In some embodiments, at least 30% of the residues of the charge component are positively charged at pH 7.0.

In various embodiments, at least 10% of the residues of the charge component are negatively charged at pH 7.0 and at least 10% of the residues of the charge component are positively charged at pH 7.0. In various embodiments, at least 12.5% of the residues of the charge component are negatively charged at pH 7.0 and at least 12.5% of the residues of the charge component are positively charged at pH 7.0. In various embodiments, at least 15% of the residues of the charge component are negatively charged at pH 7.0 and at least 15% of the residues of the charge component are positively charged at pH 7.0. In various embodiments, at least 20% of the residues of the charge component are negatively charged at pH 7.0 and at least 20% of the residues of the charge component are positively charged at pH 7.0. In various embodiments, at least 25% of the residues of the charge component are negatively charged at pH 7.0 and at least 25% of the residues of the charge component are positively charged at pH 7.0.

In some embodiments, at least 10% of the residues of the charge component are histidine. In various embodiments, at least 12.5% of the residues of the charge component are histidine. In particular embodiments, at least 15% of the residues of the charge component are histidine. In several embodiments, at least 20% of the residues of the charge component are histidine. In some embodiments, at least 25% of the residues of the charge component are histidine.

In some embodiments, at least 10% of the residues of the charge component are selected from aspartate and glutamate. In various embodiments, at least 12.5% of the residues of the charge component are selected from aspartate and glutamate. In particular embodiments, at least 15% of the residues of the charge component are selected from aspartate and glutamate. In several embodiments, at least 20% of the residues of the charge component are selected from aspartate and glutamate. In some embodiments, at least 25% of the residues of the charge component are selected from aspartate and glutamate.

Any combination of the above may apply. For example. at least 12.5% of the residues of the charge component may be histidine and at least 12.5% of the residues of the charge component may be selected from aspartate and glutamate. In another example, at least 15% of the residues of the charge component may be histidine and at least 20% of the residues of the charge component may be selected from aspartate and glutamate.

In various embodiments, the charge component may include fixed positive charge conferred by lysine and/or arginine residues.

In several embodiments, the charge component comprises one or more aromatic residues forming Cation-pi bonding interactions, such as Tryptophan, Tyrosine and Phenylalanine.

In some embodiments, the charge component is a polypeptide comprising between 2 and 8 histidine residues and between 2 and 8 aspartate and/or glutamate residues. In various embodiments, the histidine residues are clustered in a first group and the aspartate and/or glutamate residues are clustered in a second group. The clustering of the groups may be discrete and entirely non-overlapping, or may be substantially clustered, i.e. the groups may be mostly separate but with some residues overlapping.

In several embodiments, the histidine residues are interspersed with the aspartate and/or glutamate residues. The histidine residues may be fully interspersed with the aspartate and/or glutamate residues, i.e. they are evenly distributed, or the histidine residues may be partially interspersed with the aspartate and/or glutamate residues, i.e. some of the histidine residues are evenly distributed with the aspartate and/or glutamate residues and some of the histidine residues are clustered. Similarly, the aspartate and/or glutamate residues may be fully or partially interspersed with the histidine residues.

Tuning of the relative quantity of positive dissociating residues and negative dissociating residues in the charge component may give an excess charge to the charge component and consequently also to the overall construct. Moreover, as the positive dissociating residues may be histidine possessing a pKa that is close to neutral, this residue may be driven to switch between neutral and positive by raising the environmental pH above this near neutral pKa, and a number of histidines in the charge component will dissociate sequentially and progressively as the pH approaches the pKa and passes it. In an example, if the charge component possesses a relatively greater number (such as double) of histidine or near-neutral dissociating positive residues than counterpart negative residues with a much lower pKa (such as for asparagine), then at the pKa of the near-neutral dissociating residues, half will be dissociated giving them a positive charge, and half will be neutral, and the collective charge of the positively charged half will be cancelled by the collective opposite charge of the negative residues leaving the group neutral and having no net effect on the construct at this pH.

Increasing the relative ratio of neutral/positive residues to negative residues will give a higher positive charge at the same pH, and the overall neutral point of the charge component (i.e. the pI) will be shifted to a lower pH, and vice versa. This in turn confers additional positive or negative charge to the overall construct at a given pH as required, thus shifting the construct's overall pI. An example of the charge component shifting the relative pH of the mechanism of the affinity binding pair would be that having a charge component with an excess of histidines and a deficit of aspartates would give the construct a higher overall charge at any given pH, and shift the global pI to a higher pH. As the binding of the affinity pair is dependent on individual copies of the construct coming together, their propensity for binding each other is enhanced by a lack of similar excess charge as these are self-repulsive, i.e. the pI, therefore an increase of the pI would raise the overall transition pH of the affinity binding pair to a higher value as well.

The lock component and the key component are specific affinity binding partners, meaning that the lock component and the key component can bind to each other in a highly specific manner that excludes non-specific binding. The binding interaction is reversible, meaning that the lock component and key component will bind in one condition, and will dissociate in another condition. Preferably, the binding interaction is reversible under changes to the solution environment. More preferably, the binding interaction is reversible under changes to the solution pH.

The lock and key component binding partners provide the advantage that multiple copies of the construct can be bound together into a chain, polymerising into a macroscopic globule/gel. The chaining may be linear or branched. The binding may occur through covalent bonds or reversible partial bonds driven by electrostatic interaction. This allows polymerisation of the construct and subsequent separation to purify a target compound.

In some embodiments, the composition further comprises a target compound. The target compound is the compound or molecule that it is desired to purify from a solution, wherein the solution may comprise contaminants. The target compound may be free in solution.

The target compound may be a specific affinity binding partner to the key component. In these embodiments, the lock component and the target compound are both specific affinity binding partners of the key component, i.e. the lock component and the target compound both contain sub-domains that the key component specifically binds to. This provides the advantage that the target compound is able to bind to the construct by the same affinity lock and key system, guaranteeing specificity in incorporation with the construct, and also specificity of the polymerisation. In some embodiments, the lock component and the target compound have different structures. In various embodiments, the key component binds to the lock component and the target compound via different binding mechanisms. Preferably, the lock component and the target compound have the same structure or a sub-domain with the same structure. Preferably, the key component binds to the lock component and the target compound via the same binding mechanism.

In some embodiments, the lock component is modified such that the binding of the key component to the lock component is different to the binding of the key component to the target compound. For example, the lock component may be modified to increase or decrease the binding affinity between the key component and the lock component. The modification may increase or decrease the binding affinity in particular environmental conditions, such as in changing pH. The lock component may be truncated. The modification may be selected from truncation, individual residue changes at the binding site intended to modify affinity, individual residue changes intended to modify charge, changes of size of residues (e.g. from large to small and vice versa), changes to overall globular flexibility (e.g. by the addition/removal of disulfide bonds and/or salt bridges), etc.

In some embodiments, in a third environmental condition the charge component imparts a third overall charge to the first construct, the lock component binds to its binding partner on a second construct, and the key component binds to its binding partner on a third construct, and the target compound remains unbound.

In some embodiments, the third environmental condition is a third pH. In particular embodiments, the third pH may be more acidic than the first and second pH. In various embodiments, the third pH is an acidic pH and the first and second pH are neutral or near-neutral pH. In several embodiments, the third pH is at least 1.5 pH units lower than the second pH and the first pH. In some embodiments, the third pH may be an intermediate pH between the first and second pH.

The third pH may provide a condition where the constructs polymerise to form a polymer and the target compound remains unbound and free in solution, allowing the target compound to be separated from the constructs.

The lock component may be any molecule that forms a specific affinity binding pair with the key component. The lock component may be a peptide, polypeptide, nucleic acid, organic polymer, organic compound, synthetic polymer or synthetic compound. In various embodiments, the lock component is a polypeptide. In certain embodiments, the lock component is an antibody, antibody fragment or antibody mimetic.

The key component may be any molecule that forms a specific affinity binding pair with the lock component. In embodiments where the lock component comprises the target compound, the key component may be any molecule that forms a specific affinity binding pair with the target compound. The key component may be a peptide, polypeptide, nucleic acid, organic polymer, organic compound, synthetic polymer or synthetic compound. In various embodiments, the key component is a polypeptide. In certain embodiments, the key component is selected from a surface cell wall protein A, a surface cell wall protein L, a surface cell wall protein G, an antibody and an antibody mimetic. Preferably, the key component is a surface cell wall protein.

The target compound may be any molecule to be purified. The target compound may be a peptide, polypeptide, nucleic acid, organic polymer, organic compound, synthetic polymer or synthetic compound. In some embodiments, the target compound is a polypeptide. In certain embodiments, the target compound is an antibody or antibody fragment. In particular embodiments, the target compound is a polypeptide that is incorporated in a larger quaternary structure, such as a bacteria or virus or a membrane-bound protein on the surface of a cell.

In particular embodiments of the invention, the lock component and/or the target compound is an antibody or an antibody fragment and the key component is selected from a surface cell wall Protein A, a surface cell wall Protein L and a surface cell wall Protein G.

The lock component and the key component are separated on the construct such that they are unable to form a binding pair within the same construct. In other words, the lock component cannot bind a key component within the same construct. However, a lock component may bind a key component within a distinct construct and a key component may bind a lock component within a distinct construct. The skilled person will recognise that the “distinct construct” may be an identical copy of the construct, a substantially identical copy of the construct, or a different construct with the same essential elements.

In some embodiments, the construct may further comprise one or more key components, and/or one or more lock components. The additional key component(s) may be identical or substantially identical to the key component. The additional key component(s) may form a specific affinity binding pair with the lock component and/or target compound. The additional key component(s) may be modified to exhibit different properties to the key component, such as binding affinity to the lock component and/or target compound. The additional key component(s) may be separated from their binding partners within the same construct such that they are unable to form a binding pair within the same construct.

The additional lock component(s) may be identical or substantially identical to the lock component. The additional lock component(s) may form a specific affinity binding pair with the key component. The additional lock component(s) may be modified to exhibit different properties to the lock component, such as binding affinity to the key component. The additional lock component(s) may be separated from their binding partners within the same construct such that they are unable to form a binding pair within the same construct.

The additional key components and/or additional lock components provide the advantage that polymerisation of the construct can form polymers that are branched and have excess capacity for binding to target compounds.

In some embodiments, the first construct further comprises a second key component, wherein the lock component and the second key component are specific affinity binding partners, and wherein the lock component and the second key component are separated such that they are unable to form a binding pair within the same construct.

In some embodiments, the first construct further comprises a second lock component, wherein the second lock component and the key component are specific affinity binding partners, and wherein the second lock component and the key component are separated such that they are unable to form a binding pair within the same construct.

In some embodiments, the first construct further comprises a second key component, and the composition further comprises a target construct comprising a target compound, wherein the target compound is a specific affinity binding partner to the first key component and the second key component.

In some embodiments, the components are separated by rigid interlinks between neighbouring components. The interlink components may link molecular components, preferably through permanent covalent bonds, preferably having two ends linking two molecular components with one at each end, and preferably maintaining a relatively rigid shape holding the two linked molecular components rigidly away from each other.

The rigid interlinks may give the construct a linear structure which prevents components from folding over and binding to other components in the same construct. For example, the lock component and the key component may be separated by a rigid interlink that prevents the lock component and the key component forming a binding pair within the same construct. In embodiments where the lock component comprises a target compound, the target compound and the key component may be separated by a rigid interlink that prevents the target compound and the key component forming a binding pair within the same construct.

In embodiments where there are multiple lock components, and/or multiple key components, and/or multiple target compounds, the components may be separated by rigid interlinks that prevent affinity binding pairs binding within the same construct.

One skilled in the art would be able to identify the interlinks necessary to give the construct a linear structure in order to prevent binding pairs occurring within the same construct (key-lock pair, additional key-lock pair, key-additional lock pair, additional key-additional lock pair).

There is also provided a method of purification of a target compound, comprising the steps:
i) incubating a composition as previously described herein and a sample comprising a target compound at a first environmental condition, wherein the first construct binds to the target compound, and the first construct and the second construct bind together and polymerise to form a polymer;
ii) separating the polymer, wherein the target compound remains bound to the polymer;
iii) incubating the polymer at a second environmental condition, wherein the target compound dissociates from the polymer, and the first construct and the second construct dissociate and monomerise;
iv) repeating steps i to iii for between 1 to 200 cycles;
v) separating the target compound from the first construct and the second construct.

The embodiments described in relation to the composition above equally apply to this method, and vice versa.

In the above method, the “first construct” and the “second construct” refer to the monomeric constructs. The “polymer” refers to the polymerised constructs. References to the target compound being bound to the polymer also refer to the target compound being bound to individual constructs that have been polymerised.

The first incubation step may be any length of time suitable for the constructs to polymerise. This step may be the length of time for a bulk polymer to become visible to the naked eye in solution. This step may be between 1 s to 48 h, 30 s to 24 h, 1 min to 16 h, 5 min to 12 h, 20 min to 8 h, 40 min to 4 h, 1 h to 2 h.

In some embodiments, the environmental condition is selected from environmental pH, temperature, pressure, acoustic vibration, magnetic field density, electric field density, electromagnetic excitation, local component concentration, and the presence or absence of a co-enzyme or cofactor or similar activator compound. Preferably, the environmental condition is pH, i.e. the pH of a solution comprising the composition.

Mixing may occur during the first incubation step. Mixing may occur throughout the whole first incubation step or part of the first incubation step. Mixing may be by any suitable means including magnetic stirring, vortex, mechanical stirring (e.g. via impellers), bubble mixing, sonication, manual stirring, inversion and/or shaking.

Mixing provides the advantage that any impure solutes or particles that were trapped in the gel are released into the new solution environment.

The method may further comprise a step of adjusting the environmental condition of the solution. This step may involve adjusting the environment to the first environmental condition. This step may occur before, during or after the first incubation step. Preferably, this step occurs before the first incubation step. In some embodiments, this step is adjusting the pH of the solution. In various embodiments, this step is adjusting the pH of the solution to within a first pH range or to a first pH. The pH may be adjusted to about pH 7. The pH may be adjusted to a pH within the range of 5 to 9, 5.5 to 8.5, 6 to 8, 6.5 to 7.5, 6.8 to 7.2. The pH may be adjusted to pH 5, 5.5, 6, 6.2, 6.4, 6.6, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9 or 9.

The pH may be adjusted by the addition of an acidic solution or an alkaline solution. The acidic solution or alkaline solution may be inside the desired pH range. The acidic solution or alkaline solution may be buffered or may not be buffered. The acidic solution or alkaline solution may be selected from sodium acetate, acetic acid, sodium citrate, citric acid, disodium phosphate, MES, Tris, disodium phosphate, sodium phosphate, hydroxides (e.g. sodium hydroxide), tricine, bicine, CHES or a mixture thereof.

Mixing may occur during the pH adjustment step. Mixing may occur throughout the whole pH adjustment step or part of the pH adjustment step. Mixing may be by any suitable means including magnetic stirring, vortex, mechanical stirring (e.g. via impellers), bubble mixing, sonication, manual stirring, inversion and/or shaking. In order to reduce external contamination this would preferably be performed using a magnetic stirrer in a sealed unit.

Mixing provides the advantage that any impure solutes or particles that were trapped in the gel are released into the new solution environment and relatively diluted by that volume.

The separation step may be any suitable size capture technique appreciated by one skilled in the art that would separate a polymerised/bulk polymer from smaller contaminants. The separation step may be a coarse separation or a fine separation. Preferably, the separation step is a coarse separation. In various embodiments, the separation step is sifting, filtration, microfiltration, ultrafiltration, centrifugation or size exclusion. Preferably, the separation step is a coarse grained sift where the polymer is large enough that a finer separation solution or differential separation solution such as centrifugation are not required.

The separation step may take place within the same vessel or within a different vessel. The separation step may retain the polymerised polymer and remove contaminants from the solution. The contaminants may be drained away. The polymerised polymer may be separated into a clean vessel, leaving the contaminants in the initial vessel.

The second incubation step may be any length of time suitable for the polymer to monomerise into the monomeric constructs. This step may be the length of time for a bulk polymer to become invisible to the naked eye in solution. This step may be between 1 s to 48 h, 30 s to 24 h, 1 min to 16 h, 5 min to 12 h, 20 min to 8 h, 40 min to 4 h, 1 h to 2 h.

Mixing may occur during the second incubation step. Mixing may occur throughout the whole second incubation step or part of the second incubation step. Mixing may be by any suitable means including magnetic stirring, vortex, mechanical stirring (e.g. via impellers), bubble mixing, sonication, manual stirring, inversion and/or shaking. In order to reduce external contamination this would preferably be performed using a magnetic stirrer in a sealed unit.

Mixing provides the advantage that any impure solutes or particles that were trapped in the gel are released into the new solution environment.

The method may further comprise a step of adjusting the environmental condition of the solution. This step may involve adjusting the environment to the second environmental condition. This step may occur before, during or after the second incubation step. Preferably, this step occurs before the second incubation step. In some embodiments, this step is adjusting the pH of the solution. In various embodiments, this step is adjusting the pH of the solution to within a second pH range or to a second pH. The pH may be adjusted to about pH 4. The pH may be adjusted to a pH within the range of 2 to 6, 2.5 to 5.5, 3 to 5, 3.5 to 4.5. The pH may be adjusted to pH 2, 2.5, 3, 3.4, 3.6, 3.7, 3.8, 3.9, 4, 4.1, 4.2, 4.3 4.4, 4.5, 4.6, 4.7, 4.8, 4.9, 5, 5.5 or 6.

The pH may be adjusted by the addition of an acidic solution. The acidic solution may be inside the desired pH range. The acidic solution may be buffered or may not be buffered. The acidic solution may be selected from sodium acetate, acetic acid, sodium citrate, citric acid, disodium phosphate, MES, or a mixture thereof.

The method may further comprise a step of adjusting the environmental condition of the solution. This step may involve adjusting the environment to the first environmental condition. This step may occur after the second incubation step or after the step adjusting the environmental condition to the second environmental condition. In some embodiments, this step is adjusting the pH of the solution. In various embodiments, this step is adjusting the pH of the solution to within a first pH range or to a first pH. The pH may be adjusted to about pH 7. The pH may be adjusted to a pH within the range of 5 to 9, 5.5 to 8.5, 6 to 8, 6.5 to 7.5, 6.8 to 7.2. The pH may be adjusted to pH 5, 5.5, 6, 6.2, 6.4, 6.6, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9 or 9.

The pH may be adjusted by the addition of an alkaline solution. The alkaline solution may be inside the desired pH range. The alkaline solution may be buffered or may not be buffered. The alkaline solution may be selected from Tris, disodium phosphate, sodium phosphate, hydroxides (e.g. sodium hydroxide), tricine, bicine, CHES, or a mixture thereof.

The method may further comprise a step of adjusting the environmental condition of the solution. This step may involve adjusting the environment to a third environmental condition. This step may occur as preceding the final separation step or as part of the final separation step. In some embodiments, this step is adjusting the pH of the solution. In various embodiments, this step is adjusting the pH of the solution to within a third pH range or to a third pH. The third pH may be a pH that is more acidic than the first and second pH. The third pH may be an intermediate pH between the first pH and the second pH.

Mixing may occur during the pH adjustment step(s). Mixing may occur throughout the whole pH adjustment step or part of the pH adjustment step. Mixing may be by any suitable means including magnetic stirring, vortex, mechanical stirring (e.g. via impellers), bubble mixing, sonication, manual stirring, inversion and/or shaking. In order to reduce external contamination this would preferably be performed using a magnetic stirrer in a sealed unit.

Mixing may occur for between 1 s to 48 h, 30 s to 24 h, 1 min to 16 h, 5 min to 12 h, 20 min to 8 h, 40 min to 4 h, 1 h to 2 h.

Mixing provides the advantage that any impure solutes or particles that were trapped in the gel are released into the new solution environment.

The above steps of the method (including or excluding optional steps) may be repeated any number of times. Preferably the steps of the method are repeated for up to 200 cycles. The steps of the method may be repeated 1, 2, 4, 6, 8, 10, 12, 14, 16, 18, 20, 22, 24, 26, 28, 30, 35, 40, 50, 75, 100, 150 or 200 cycles. One skilled in the art will appreciate that the number of cycles required will depend on the contaminants in solution.

The volumes and properties (including but not limited to pH, salt concentration, ionic strength, conductivity, viscosity, presence or absence of excipient or stabilising agents or temperature) of the reagent solutions used may vary with each cycle. An increased amount of the construct or target compound (either the same or different) may be added prior to, during or after each cycle. With each cycle, contaminants are further diluted out until a satisfactory purity is obtained.

The separation step of separating the target compound from the constructs may be any suitable size capture technique appreciated by one skilled in the art, preferably a coarse-grained filtration or sifting operation. In various embodiments, the separation step is a separate affinity chromatography, combined affinity chromatography, ion-exchange chromatography or size-exclusion chromatography. In the case of separation through chromatography steps, the affinity binding of the construct may be broken to separate the construct molecules into monomers, allowing them to pass through a chromatography media alongside the target compound.

The separation step may take place within the same vessel or within a different vessel. The separation step may retain the target compound and remove the constructs from the solution. The separation step may retain the constructs and remove the target compound from the solution. The target compound may be separated into a clean vessel, leaving the constructs in the initial vessel. The constructs may be recycled to be used in the method again.

There is also provided a polymer produced by the above method.

There is also provided a nucleic acid sequence encoding the first construct of the composition as previously described herein. The first construct may be a polypeptide construct.

There is also provided a nucleic acid sequence according to SEQ ID NO: 1, a vector comprising said nucleic acid sequence, a host cell comprising the composition as previously described herein, a host cell comprising said nucleic acid sequence, and a host cell comprising said vector.

There is also provided a polypeptide comprising an amino acid sequence according to SEQ ID NO: 2.

There is also provided a polypeptide comprising an amino acid sequence according to SEQ ID NO: 6.

### BRIEF DESCRIPTION OF THE DRAWINGS

The invention will now be described in detail by way of example only with reference to the figures in which:

FIG. 1 shows visible formation of construct polymer. Polymer formation was visible by the naked eye of a prototype construct as described in Example 1, after desalting from an acidic solution to a neutral solution.

FIG. 2 shows the structural arrangement of the domains of three constructs in the N-terminal to C-terminal direction (left to right). (A) An exemplary construct from Examples 1 and 5. (B) An exemplary construct from Example 2. (C) The comparative construct from Example 3.

FIG. 3 shows SDS-PAGE gels of Example 1 construct with host cell (bacterial) proteins and spiked antibody. A full breakdown of the samples in each lane is provided below. Two SDS-PAGE gels of samples containing construct with host cell (bacterial) protein spiked with antibody after polymerisation at pH 7 and 8 (A) and re-monomerisation at pH 3.6 (B) as described in Example 1. In both gels lane 1 is the protein ladder with sizes (in kDa) as shown in FIG. 3, bands corresponding to the construct are circled (as expected at the 54 kDa region) and antibody bands are labelled in gel A and indicated in the rectangular box in gel B (at the expected size of 150 kDa). In A, the construct and majority of host cell protein is collected in the polymer and disappears at pH 7 and 8 along with spiked antibody. Some excess antibody appears to remain at the higher concentration at pH 8 (A9 and A10). Upon resuspension at pH 3.6, the monomerised antibody and construct re-appear. Contaminants also re-appear but at reduced intensity showing a lower concentration due to the dilution effect between the pH cycles.

### Gel A:

1. Protein ladder with relevant sizes labelled (in kDa)
2. Monomerised sample prior to pH change with no antibody spike
3. Sample at pH 7 for 15 minutes at low antibody concentration
4. Sample at pH 7 for 45 minutes at low antibody concentration
5. Sample at pH 8 for 15 minutes at low antibody concentration
6. Sample at pH 8 for 45 minutes at low antibody concentration
7. Sample at pH 7 for 15 minutes at high antibody concentration
8. Sample at pH 7 for 45 minutes at high antibody concentration
9. Sample at pH 8 for 15 minutes at high antibody concentration
10. Sample at pH 8 for 45 minutes at high antibody concentration

### Gel B:

1. Protein ladder (same as in A)
2. Monomerised sample prior to pH change with spiked antibody at high concentration
3. Sample from equivalent lane in A but polymer pellet re-suspended at pH 3.6
4. Sample from equivalent lane in A but polymer pellet re-suspended at pH 3.6
5. Sample from equivalent lane in A but polymer pellet re-suspended at pH 3.6
6. Sample from equivalent lane in A but polymer pellet re-suspended at pH 3.6
7. Sample from equivalent lane in A but polymer pellet re-suspended at pH 3.6
8. Sample from equivalent lane in A but polymer pellet re-suspended at pH 3.6
9. Sample from equivalent lane in A but polymer pellet re-suspended at pH 3.6
10. Sample from equivalent lane in A but polymer pellet re-suspended at pH 3.6

FIG. 4 shows an embodiment of the invention where the target compound doubles up as a lock component and exists as a separate entity to the construct but in the same mixture. The target compound can be any compound that features the appropriate lock component as part of its structure, whether that be natural or modified or a fused addition. The construct is composed of a key component joined by an interlink to a sub-lock component that may also bind to key components, which in turn is joined by an interlink to another key component, which in turn is joined by an interlink to a charge component. The figure shows how subunits of the construct can be alternately and reversibly switched between a bulk polymer form incorporating the target compound, and a monomer form in free solution by switching between a first and second environmental condition. As the target and sub-lock can be designed with slightly different propensities for binding the key component, a careful selection of a third environmental condition balanced between the first two can allow the sub-lock to bind the key component while the target does not, allowing formation of a polymer of the construct only with many branches bound by multiple lock/key bonds, leaving the separate target in free solution.

FIG. 5 shows SDS-PAGE gels of the constructs of Examples 2 and 3. The left panel shows the protein ladder used in the SDS-PAGE gels for reference. Each sample has been prepared for SDS PAGE by addition of 1 in 4 SDS PAGE sample buffer, followed by denaturation through heating at 90 degrees Celsius for 3 minutes. Thermofisher Scientific NuPage SDS PAGE Bis/Tris precast gels were bathed in MES SDS running buffer 1× and run at 200V for 20-30 minutes as required. Afterwards gels were fixed and stained using Coomassie/Methanol staining solution and heated, and were destained using water. The right panel shows the SDS-page gel. A full breakdown of the samples in each lane is provided below.
1. Protein ladder with relevant sizes labelled (in kDa)
2. Raw antibody diluted to 0.025g/L
3. Additional tested construct “4”
4. Additional tested construct “4”
5. Additional tested construct “4”
6. Additional tested construct “5”
7. Additional tested construct “5”
8. Additional tested construct “5”
9. ‘Supernatant’ of Antibody-spiked sample (0.025 g/L) plus Example 2 construct, buffer exchanged into pH 3.6 (control condition) and spun down
10. Supernatant of Antibody-spiked sample (0.025 g/L) plus Example 2 construct, buffer exchanged into pH 7.0 and spun down
11. Remonomerised gel pellet of Antibody-spiked sample (0.025 g/L) plus Example 2 construct, buffer exchanged into pH 7 (control condition), spun down, separated and re-dissolved in 5 0mM Sodium acetate pH 3.6
12. Supernatant' of Antibody-spiked sample (0.025 g/L) plus Example 3 construct, buffer exchanged into pH 3.6 (control condition) and spun down
13. Supernatant of Antibody-spiked sample (0.025 g/L) plus Example 3 construct, buffer exchanged into pH 7.0 and spun down
14. Remonomerised gel pellet of Antibody-spiked sample (0.025 g/L) plus Example 3 construct, buffer exchanged into pH 7 (control condition), spun down, separated and re-dissolved in 50 mM Sodium acetate pH 3.6

FIG. 6 shows Dynamic Light Scattering (DLS) data for the construct of Example 2 (A) and the construct of Example 3 (B) in the pH 3.6 and pH 7 conditions.

### DETAILED DESCRIPTION OF THE INVENTION

### Example 1

In an example, the polymer has the following structure:
Key-lock-key-charge component

The target compound is produced separately and remains free in solution.

In an embodiment of the invention the target compound also doubles up as the affinity “lock” component and is produced separately and remains in free solution. On a separate construct molecule, a key component is bound by an interlink to a sub-lock component that binds to the same key component as the target compound also by a specific affinity binding mechanism as the target compound which in turn is bound by an interlink to another copy of the same key component which in turn is bound by an interlink to a charge component.

In the system of this embodiment at a high condition such as a high pH, the lock components bind the key components and the charge component has a strong positive charge conferring the construct an overall positive charge. In this state either key component binds to the sub-lock component from another molecule and either may instead bind to the target compound, and the rigid linker between the lock and key components on an individual construct prevent them from folding over and binding each other. In this way each key component from each construct will only bind sub-lock components from other constructs or target compounds freely floating in the solution environment. As there are two or more key components they may bind lock/target components from more than one construct forming a branching chain and also bind target compounds. Optionally the construct may also include multiple copies of the sub-lock and therefore branching may occur in this way as well. Altering the relative number of copies of key/sub-lock affects the propensity for branching. This chain will grow to a length and branches such that up to all the constructs in the solution may join into a single branched polymer chain, or a larger number of smaller polymer chains may form still of a long length and many branches. The very large size of these branched polymers will cause them to form a bulk macroscopic mass, with the individual component sub-units still solvated by the solvent, but the bulk too massive to remain a free flowing solute and thus should form a type of gel. It may be large enough that it can be physically filtered by a coarse grain filter or sift. Using such a coarse grain filter or sift allows the removal of the construct polymer complex or complexes with bound target compounds from the bulk solvent and other solvated or suspended impurity solutes or particles, and the gel may be deposited in a secondary solution which may possess a different chemical environment, volume, and impurity mixture, and so this step can alter the concentration and relative purity and re-exchange into a new solution environment, and thereby also induce a shift in the chemical properties of the constructs making up the gel. At a lower condition, such as a slightly lower pH, the charge component may become neutrally charged. At a lower condition still, the charge component may become negatively charged. Separately, at a lower condition than the first, the overall construct may become neutrally charged predominantly as a result of the shifting charge of the charge component, and at a lower condition still the overall construct may become negatively charged, again predominantly as a result of the shifting charge of the charge component. Separately, at a lower condition than the first, the binding rate of the target/lock component for the key components may reduce such that the target compounds that were previously joined to the polymer complexes now dissociate and are released into the new solution environment while the sub-lock is engineered to have a higher affinity for the key and retains its binding and therefore the binding of the constructs into polymer complexes and in this state a course filter or sift could optionally be used to filter the gel composed of complexes out of the solution environment leaving the target compound behind. At a lower condition still, the sub-lock components lose their affinity for the key components and the construct complexes separate into monomers once again. Throughout by taking advantage of the different controlled state of the charge component the construct may be further separated or purified from the solution environment conventional purification techniques such as chromatography, including ion exchange, Hydrophobic interaction, a different kind of affinity, a mixed mode or any other. By cycling the constructs between polymer and monomer states the polymerised construct including the target compounds may be sifted, redeposited in a new solution, re-monomerised and mixed with the new environment, and re-polymerised repeatedly. The re-monomerisation may occur immediately in the new solution with each re-deposition as the chemical environment may be pre-prepared such that it immediately induces monomerisation. Or a chemical additive such as acid may be added at a later time to induce monomerisation as required. At this time, mixing of the solution allows any impure solutes or particles that were trapped in the gel to be released into the new solution environment. Re-polymerisation may then be induced by adding a chemical additive to the solution environment, such as an alkali, and the amount that's added can be controlled such that the re-polymerisation occurs with different levels of overall charge of the construct and charge of the charge component. Polymerisation has the effect of preferentially concentrating the construct molecules in a condensed region of space in the solution environment, leaving the remaining bulk of impurities fee in the solution environment, and controlling the charge of the construct during this polymerisation may be used to additionally reject sets of impure molecules via charge repulsion. Thus, with each cycle of polymerisation, sifting, re-deposition, re-monomerisation and mixing the purity of the product may be successively increased, and by changing the charge during polymerisation, additional classes of impurities may be rejected. At a final cycle the solution environment can be adjusted to the intermediate state where the sub-lock binds the key and the target compound does not, forming a polymer and in a final sifting process the construct may be separated leaving the target behind and in a pure state. The construct may be discarded, or may be reused in a new batch of impure feed containing the target compound. In this way the target compound and construct may also be synthesised in the first instance separately and brought together for a purification sequence as described.

### Example 2

In this example, a variant of the construct incorporates:
two copies of a Protein L B-domain affinity binding partner;
a Kappa Light chain antibody fragment as a counterpart;
charge components located at the linkers and C-Terminal end (in this case split over space into the acidic groups at the linkers and Histidine Base group at the end).

The order of components (5′ to 3′), as shown in FIG. 2B, was as follows: Protein L B-domain, negative linker, Protein L B-domain, negative linker, Kappa Light Chain, positive charge component.

The expressed construct had a polypeptide sequence according to SEQ ID NO:6.

The construct was expressed in a BL21(DE3) E. coli cell line by means of a pET vector in a culture that is grown using an LB-growth medium and induced to express via IPTG. The resulting cells were pelleted in a centrifuge to concentrate them and remove the culture media. B-PER TH cell lysis reagent (Thermofisher Scientific) was added to release the cell contents and DNase I (Thermofisher Scientific) was added to prevent precipitation into an insoluble fraction caused by DNA in later steps. 6M Guanidinium Hydrochloride was added to further prevent precipitation into an insoluble fraction in later steps and stop the proteolytic action of proteases. The mixture was passed through a PD10 buffer exchange column (Cytiva) to exchange out the B-PER™/Guanidinium mixture and displace it into a sodium acetate solution at pH 3.6. This was the starting mixture and contained both the construct and many polypeptide and cellular impurities. At this pH, the Protein L affinity binding partners were prevented from binding to the Kappa Light chain and the construct remained in the liquid fraction in a predominantly monomeric form.

A full length human IgG antibody purification target (length approximately 145kDa) was diluted to an approximately equivalent concentration as the construct (through observation on SDS-PAGE gels), and was added to the mixture, mixed, and sampled. 37.5 μl of sample was prepared for running on SDS-PAGE directly. 2×130 μl of sample was passed through a 7 kDa MWCO buffer exchange/separation Zeba Column (Thermofisher Scientific) that was pre-equilibrated with Sodium Phosphate pH 7.0. 130 μl of sample was passed through 7 kDa MWCO buffer exchange/separation Zeba Column (Thermofisher Scientific) that was pre-equilibrated with Sodium Acetate pH 3.6 as a control. The mixture, including the full-length antibody, the construct, and many impurities of a large range of sizes (>7 kDa) were thus quickly exchanged into a pH of 7.0 and a second control lot into a well exchanged pH of 3.6 environment. The buffer exchanged effluents were mixed and allowed to incubate for 15 minutes at room temperature in this condition.

In the pH 7.0 condition, the Protein L affinity binding partners were able to bind the Kappa Light chains in copies of the construct, forming polymers of construct. The excess of Protein L allowed some copies to bind the Kappa Light chain from more than one copy of a construct, forming branches in the polymer. The excess of Protein L allowed the bulk of the copies of the construct to bind the free full length IgG antibody. The rigid linkers and close proximity prevented the Protein L and Kappa Light chains from folding back and binding within one copy of the construct.

The rigidity and orientation of the Protein L and Kappa light chains in a single unit of the construct prevented excessive formation of stable dimers. The affinity bond of the Protein L and Kappa Light chains binding in specific orientations which, together with the rigidity of the whole construct, prevented the free ends from bending around and also binding to each other.

The Protein Ls could bind at multiple sites on the antibody light chain and so multiple copies of the construct could bind to the antibody, forming a branch in the polymer. The charge groups integrated into the construct in this case gave the construct a theoretical overall pI of 7.54, and so in this buffering condition (pH 7.0) gave it a small global negative charge, and at pH 8.0 gave it a small global positive charge. This allows tuning of the electrostatic repulsion of like-charge particles by controlling the buffering pH.

In the pH 3.6 condition, the Protein L affinity binding partners were reversibly inactivated from their binding propensity for the Kappa Light chains in copies of the construct. Thus, the constructs were unable to form polymers and were unable to bind the IgG antibody and both remained free in solution.

In the pH 7.0 condition, the construct-antibody polymer formed visible particles while the 3.6 condition did not. These were both centrifuged, forming a gel-pellet of the polymer in the pH 7.0 condition, and the remaining supernatants were decanted and prepared to run on SDS-PAGE. The remaining gel-pellet was resuspended in an equivalent volume of Sodium Acetate pH 3.6 and incubated while mixing. The change in the pH environment caused the polymer to re-monomerise into liquid suspension. This was also prepared to run on SDS-PAGE.

The Gel in FIG. 5 shows the raw antibody in lane 2 with a faint antibody fragment (size between 98 and 145 kDa). The antibody spiked control sample that is buffer exchanged into pH 3.6 is shown in lane 9. The supernatant of the antibody spiked sample that is buffer exchanged into pH 7.0 is shown in lane 10. The re-monomerised gel-pellet is shown in lane 11.

Lane 9 shows the strong presence of the antibody mixed with a mixture of cellular impurities of various sizes as well as a moderate band between 38 and 49 kDa (for reference the construct is 40 kDa). Lane 10 shows that the antibody has been substantially removed from the supernatant mixture, as has the ˜40 kDa construct band. The corresponding mixture of cellular impurities of various sizes has been left behind. This indicated that the antibody had been overwhelmingly sequestered from solution as had the presence of the construct. In lane 13 (Supernatant of Antibody-spiked sample (0.025 g/L) plus Example 3 construct, buffer exchanged into pH 7.0 and spun down), the full-length antibody re-appeared and at the same apparent concentration as the original addition, demonstrating high recovery of the antibody. Furthermore, the mixture of other proteins has been significantly removed from the mixture indicating a high level of purification. Thus, the system has demonstrated product sequestration, product recovery, product buffer displacement, product concentration, general impurity removal.

### Example 3

In this example, a variant of the construct incorporates:
two copies of a Protein L B-domain affinity binding partner;
a Kappa Light chain antibody fragment as a counterpart.

The order of components (5′ to 3′), as shown in FIG. 2C, was as follows: Protein L B-domain, linker, Protein L B-domain, linker, Kappa Light Chain.

The expressed construct had a polypeptide sequence according to SEQ ID NO:7.

The construct was expressed and mixed with the same full length human IgG antibody purification target and buffer exchanged into pH 7.0 or pH 3.6 as described in Example 2 above.

In the pH 7.0 condition, the Protein L affinity binding partners were able to bind the Kappa Light chains in copies of the construct, forming polymers of construct. The excess of Protein L allowed some copies to bind the Kappa Light chain from more than one copy of a construct, forming branches in the polymer. The excess of Protein L allowed the bulk of the copies of the construct to bind the free full length IgG antibody. The rigid linkers and close proximity prevented the Protein L and Kappa Light chains from folding back and binding within one copy of the construct.

The rigidity and orientation of the Protein L and Kappa light chains in a single unit of the construct prevented excessive formation of stable dimers. The affinity bond of the Protein L and Kappa Light chains binding in specific orientations which, together with the rigidity of the whole construct, prevents the free ends from bending around and also binding to each other.

The Protein Ls could bind at multiple sites on the antibody light chain and so multiple copies of the construct could bind to the antibody, forming a branch in the polymer. Without an additional integrated charge component, the pI of the components of the construct was 4.87, and so in this buffering condition (pH 7.0) gave the construct a significant positive charge, and thus repels other like-charges.

In the pH 3.6 condition, the Protein L affinity binding components were reversibly inactivated from their binding propensity for the Kappa Light chains in copies of the construct. Thus, the constructs were unable to form polymers and were unable to bind the IgG antibody and both remained in free solution.

In the pH 7.0 condition the construct-antibody polymer formed visible particles. These were centrifuged forming a gel-pellet of the polymer and the remaining supernatant was decanted and prepared to run on SDS-PAGE. The remaining gel-pellet was resuspended in an equivalent volume of Sodium Acetate pH 3.6 and incubated while mixing. The change in the pH environment caused the polymer to re-monomerise into liquid suspension. This was also prepared to run on SDS-PAGE. Similarly, the pH 3.6 condition was also centrifuged and the supernatants prepared for running on SDS-PAGE.

The gel in FIG. 5 shows the antibody mixed with the cellular contents and exchanged into Sodium Acetate pH 3.6 in lane 12. The supernatant after adjusting and incubation at pH 7.0 is shown in lane 13. The re-monomerised gel-pellet is shown in lane 14. Lane 12 and 13 show corresponding mixes of impurities of various high and low molecular weights; lane 12 shows the significant presence of full length antibody and lane 13 shows the antibody has been significantly reduced from the supernatant mixture. Similarly, a faint band corresponding to the construct. Additionally, a fragment of the antibody (size between 98 and 145 kDa) has also been left behind. This indicates that the antibody has been significantly sequestered from solution as has the presence of the construct. In lane 14, the full-length antibody re-appears at a higher apparent concentration as the antibody remaining in the supernatant fraction in lane 13 and a significant fraction of the concentration as the original addition seen in lane 12, demonstrating substantial recovery of the antibody. Furthermore, the mixture of other proteins has been significantly removed from the mixture indicating a high level of purification. Additionally, the antibody fragments have also been significantly removed, indicating purification of even target related impurities. Thus, the system has demonstrated product sequestration, product recovery, product buffer displacement, product concentration, general impurity removal and target related impurity removal (i.e. antibody fragments).

Example 2 and Example 3 are structurally similar with both possessing two copies of Protein-L B-domain followed by identical copies of Kappa Light chain. They are differentiated by the presence of the charge component in Example 2 (a combination of Histidine residues collected at the C-terminal end with interspersed charged amino acids in this end and embedded in rigid linker regions) and the absence of the charge component in Example 3. In identical operating conditions, the inventors could therefore observe the difference induced by the charge component. There was an observed difference in relative apparent sequestration levels between Examples 2 and 3 as seen in the higher level of antibody separation observed from lane 10 to 11 in Example 2 and lane 13 to 14 in Example 3. There was also apparently differentiated levels of relative removal of the pattern of bands of impurities in the mid-sized range (14-28 kDa). These indicated that the separation behaviour was differentiated between the two versions of the construct, allowing the inventors to conclude that the presence of the charge component amino acids was able to successfully and tuneably modulate the separation functions of the construct.

### Example 4

To investigate the polymerisation process further, materials from Examples 2 and 3 were examined using a dynamic light scattering (DLS) device. Some antibody spiked resolubilised pellet of each, both in 50 mM Sodium Acetate pH 3.6 buffering conditions, were split into two; in the first 32.5 μl of sample was diluted with a further 22.5 μl of 50 mM Sodium Acetate buffer pH 3.6 maintaining the original pH and acting as a control, while in the second 32.5 μl of sample was diluted with a further 22.5 μl of 400 mM Sodium Phosphate pH 9, giving a new combined pH of 7.0. These were left to incubate for 30 minutes at room temperature and were measured by a Malvern Instruments APS2000 Zetasizer. FIG. 6A shows the DLS data for the construct of Example 2. The data clearly shows smaller particle sizes centred around a diameter of 600 nm in the pH 3.6 condition, and also shows larger particle sizes centred around a diameter 2200 nm in the pH 7.0 condition. These peaks clearly demonstrate polymerisation is occurring when the pH is increased to pH 7.0. The third species on the far left of the DLS data shows a third smaller species that may be either a fragment of the polymerised body broken away to form a smaller particle, or potentially an antibody or antibody fragment. Similarly, in FIG. 6B (showing the data for the construct of Example 3), a distribution of smaller particles is observed centred at a diameter of 200 nm in the pH 3.6 condition, and a distribution of larger polymerised particles centred at a diameter of 1600 nm in the pH 7.0 condition. Similar to the construct of Example 2, a smaller peak can be seen at the far left which could also represent a fragment of either the polymerised form or an antibody or antibody fragment. In this case this smaller fragment also corresponds to the smaller particle size observed in the pH 3.6 condition. As this peak is also substantially smaller in volumetric occupancy than both the large polymer peak in the pH 7.0 condition and the smaller peak in the pH 3.6 condition, this indicates that the smaller peak is being consumed to form the larger polymer when the pH is increased from 3.6 to 7.0.

### Example 5

The construct used in this prototype example was designed to purify antibodies from a crude mix of cells. This prototype was a protein construct that comprises a wild-type B-domain, connected via a short amino acid linker to an Fc domain connected via a short amino acid linker to a second Fc domain (FIG. 2A).

The expressed construct had a polypeptide sequence according to SEQ ID NO:2.

The DNA sequence (SEQ ID NO:1) of the construct was cloned into a pET100 cloning vector. Unless otherwise stated, reagents for the following steps were purchased from

Thermo Fisher Scientific. Large volumes of media and consumables were sterilised using an autoclave. Smaller volumes (up to 50 ml) of reagents were sterilised via syringing through 0.2 μm filters.

The pET100 plasmid containing the prototype construct was transformed into E. coli One Shot® BL21 (DE3) chemically competent cells following the manufacturers recommended protocol. A volume of 100 μl/plate of transformation reaction was grown overnight at 37° C. on LB-agar plates containing ampicillin at 100 μg/ml to select transformants.

Single colonies were picked and used to inoculate overnight culture containing LB media supplemented with 100 μg/ml of ampicillin. These were grown at 37° C.

The overnight cultures were used to inoculate 200 ml of LB media containing 100 μg/ml ampicillin in 0.5 L shake flasks. These were grown at 37° C. for 2 hours and agitation at 200 RPM.

Production of the prototype was induced via the addition Isopropyl β-d-1-thiogalactopyranoside (IPTG) to a final concentration of 0.3 mM. Cells were grown for a further 6 hours at 37° C. with agitation at 200 RPM.

Cells were harvested via centrifugation using a benchtop centrifuge at 5000×g for 10 minutes and the pellets stored at −80° C.

For extraction, cell pellets were defrosted at room temperature and B-PER TH Bacterial Protein Extraction Reagent was used to lyse the cells according to the manufacturer's protocol. For each 0.5 g of cell pellet 2 ml of B-PER reagent were added.

6M Guanidine hydrochloride (Gd-HCl) was added to the lysed cells to unfold any construct that had expressed and misfolded into inclusion bodies.

Construct was re-folded by taking the denatured sample and passing it through a disposable PD-10 desalting column (GE Healthcare) equilibrated with sodium acetate at pH 3.6 to keep the construct in its monomeric form but with the majority of host-protein contaminants present.

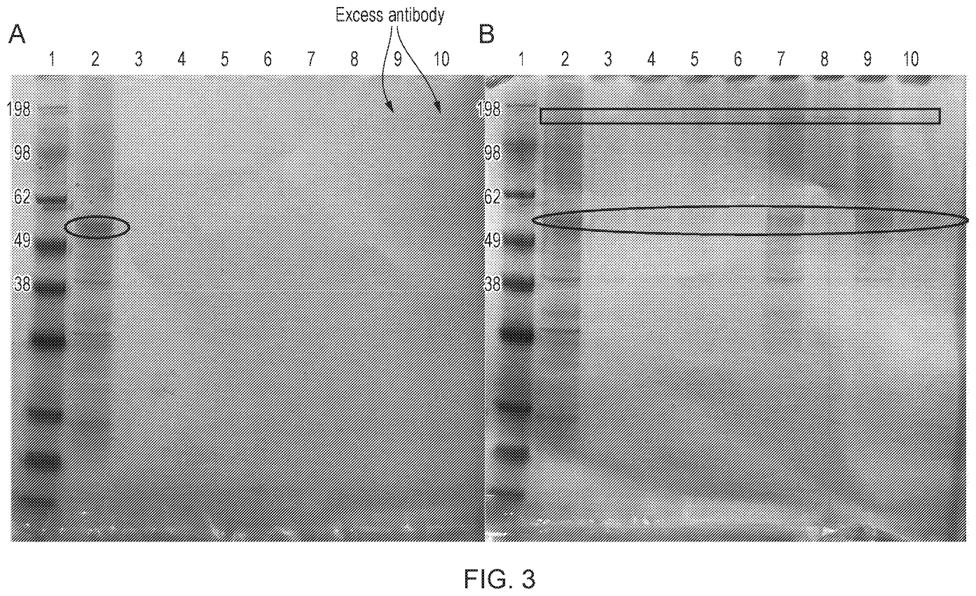

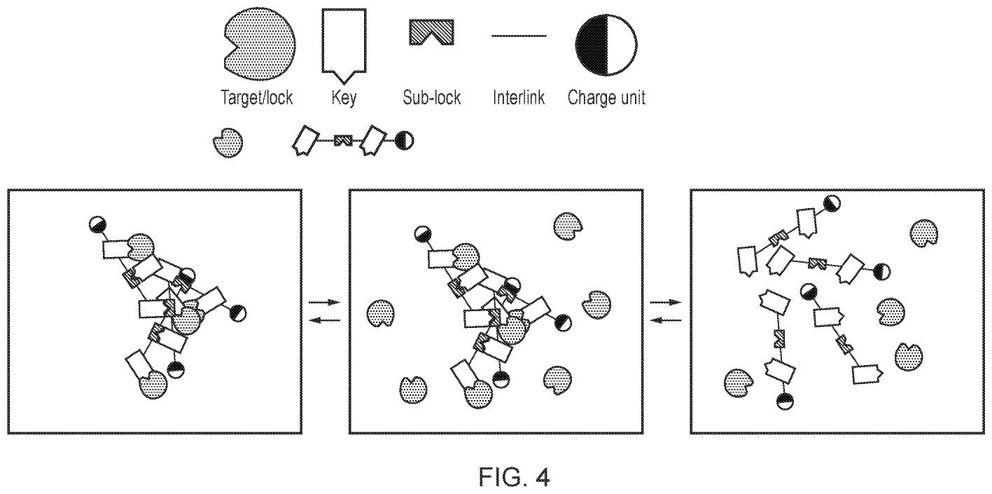

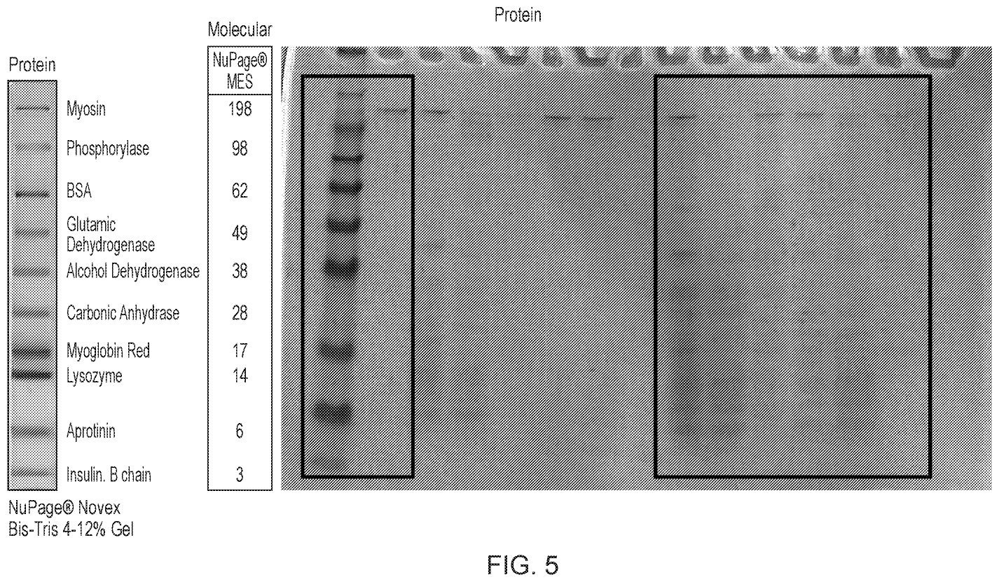

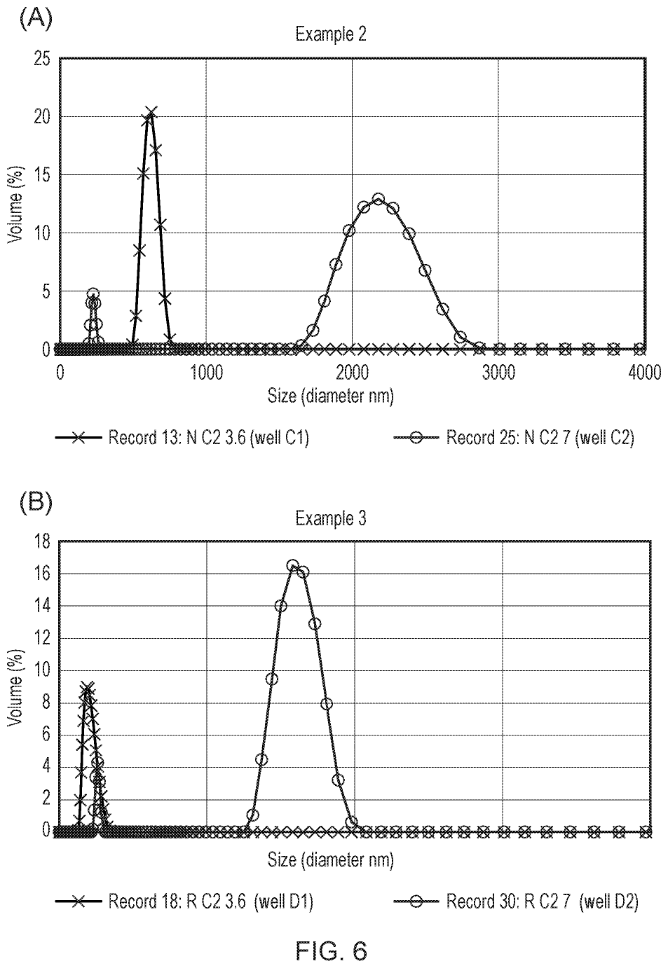

Pure antibody was spiked into the mix containing the construct. Analysis in SDS-PAGE showed that contaminants present in the sample masked the antibody band compared to the clean sample (data not shown). The pH adjusted upwards to pH 7 and pH 8 using sodium phosphate buffer for up to 45 minutes. This was centrifuged for 2 minutes at 13000×g to collect the polymer. Both construct and antibody disappear from the soluble sample (FIG. 3A). Attempts to analyse the samples via SDS-PAGE prior to centrifugation failed due to the difficulties in pipetting the polymerised samples.

The polymer was re-monomerised by addition to the centrifuged pellet of sodium acetate at pH 3.6. Samples were analysed using SDS-PAGE and show the disappearance of construct and antibody into the polymer at pH 7 and 8 with recovery of antibody and construct and a small dilution of host protein contaminants (FIG. 3) at pH 3.6.

### Example 6

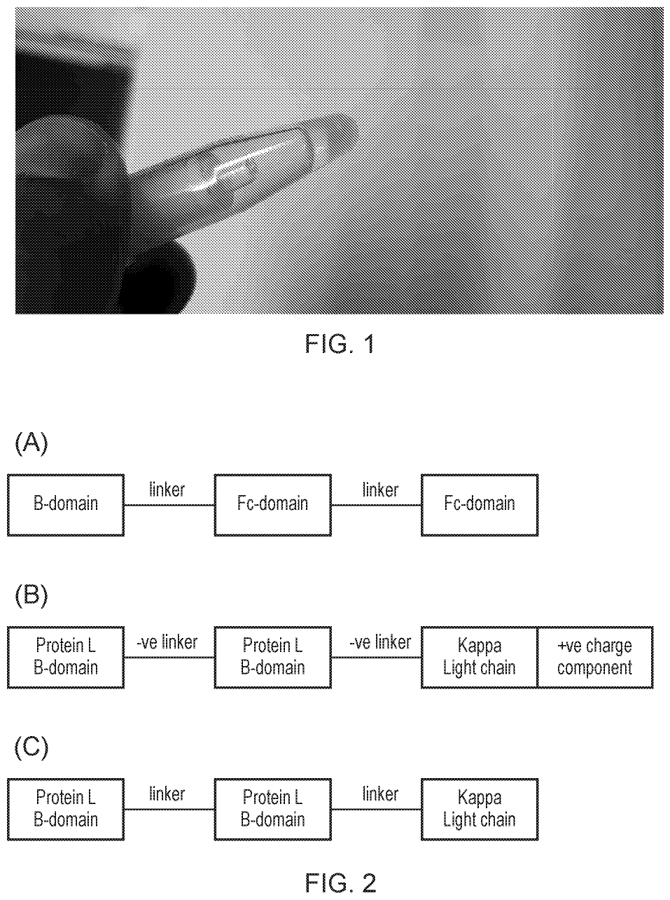

The method comprises the following steps:
1. A polymerising construct as described in the summary of description is added to a bulk sample which contains a target molecule which can be a protein or nucleic acid and a mixture of contaminants from which it needs to be separated. The addition of a protein construct can include the production of the protein construct in the same sample (either co-expressed in the same cells or in a network of different cells) as the target biomolecule. Contaminants in the sample may include (but are not limited to) one of or a mix of any of the following: host cell proteins, DNA, RNA, other nucleic acid variants, viruses, buffers, culture/growth media, lipids, insoluble components, organelles, endotoxins, exosomes, plant matter, insect tissue, bacterial cells and environmental contaminants, in vivo contaminants.
2. The sample containing both target molecule and protein construct may be left to stand or mixed using conventional means suitable for the purification scale which can include but is not limited to magnetic stirring, vortex, mechanical stirring (e.g. via impellers), bubble mixing, sonication, manual stirring, inversion, or shaking. It is left to mix or stand for a designated amount of time from 1 second to 48 hours including 30 seconds, 1 minute, 5 minutes, 20 minutes, 40 minutes, 1 hour, 4 hours, 8 hours, 12 hours, 16 hours, 24 hours or 48 hours. The pH may be adjusted to at or near neutral pH and within the range of 5-9, including pH 5, 5.5, 6, 6.2, 6.4, 6.6, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9 or 9. A polymer containing the target biomolecule and the protein construct will form (FIG. 1). This may or may not be visible depending on the scale, properties of the solutions used and properties of the sample.
3. The polymer is captured by a suitable size capture technique. This includes but is not limited to filtration, microfiltration, ultrafiltration, centrifugation, size exclusion. This can be within the same vessel or via a separate vessel/device. Contaminants are drained away whilst the target molecule remains within the captured polymer.
4. The captured biopolymer is monomerised by “washing” with an acidic solution within the pH range of 2-6, including pH 2, 2.5, 3, 3.4, 3.6, 3.7, 3.8, 3.9, 4, 4.1, 4.2, 4.3 4.4, 4.5, 4.6, 4.7, 4.8, 4.9, 5, 5.5 or 6. The acidic solution may or may not be buffered and acidic solutions may contain (but are not limited to) one of, or a mixture of sodium acetate, acetic acid, sodium citrate, citric acid, disodium phosphate or IVIES. The resulting mix may or may not be mixed using the techniques described in step 2 for a designated amount of time from 1 second to 48 hours including 30 seconds, 1 minute, 5 minutes, 20 minutes, 40 minutes, 1 hour, 4 hours, 8 hours, 12 hours, 16 hours, 24 hours or 48 hours. Contaminants that may have been captured in the biopolymer are diluted in this step.
5. The resulting solution from step 4 now contains the target biomolecule and the protein construct in monomerised form. The pH is adjusted using a suitable reagent, which may or may not be buffered and may contain (but are not limited to) one of or a mixture of Tris, disodium phosphate, sodium phosphate, hydroxides (e.g. sodium hydroxide), tricine, bicine or CHES. It is adjusted to neutral or near neutral pH and within the range of 5-9, including pH 5, 5.5, 6, 6.2, 6.4, 6.6, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9 or 9. A polymer containing the target biomolecule and the protein construct will form (FIG. 1). This may or may not be visible depending on the scale, properties of the solutions used and properties of the sample.
6. Steps 3-5 are repeated for up to 200 cycles including 1, 2, 4, 6, 8, 10, 12, 14, 16, 18 20 22, 24, 26, 28, 30, 35, 40, 50, 75, 100, 150 or 200 cycles. The volumes and properties (including but not limited to pH, salt concentration, ionic strength, conductivity, viscosity, presence or absence of excipient or stabilising agents or temperature) of the reagent solutions used can vary with each cycle and more polymerising protein construct or biomolecule target sample (either the same or different to that in step 1) can be added prior to, during or after each cycle. With each cycle contaminants are further diluted out until a satisfactory purity is obtained.
7. The final step separates the polymerising construct from the target biomolecule. This is dependent on the properties of the construct as described in element B. It may include but is not limited to a separate or combined affinity chromatography, ion-exchange chromatography, size-exclusion chromatography, proteolytic cleavage or proteolytic self-cleavage. Polymerising construct can also be recycled to be used in further batches of purification.

### Example 7

A further example system includes Camelid antibody fragments as an example of a single-domain antibody or antibody mimetic or ‘scaffold’ protein. Like human antibodies, these Camelids can be raised through an immune response or library selection against a suitable protein based antigen to achieve very high binding affinity and specificity, with the added benefit of being compact and composed of a single domain. They may be further engineered to excise further length and joined into a single polypeptide. Thus, such a programable system may be used to create a class of programmable affinity. In this example, the Camelid may be incorporated as the (Lock) affinity binding partner in the construct, with its specific antigen partner (Key) also incorporated into the construct.

Such ligand partners could include common proteins such as Lactate dehydrogenase or Glutathione-S Transferase, or more benign partners such as Albumin, or rarer proteins such as Green Fluorescent Protein (GFP). In the case of GFP, this would have the benefit of allowing optical fluorescence monitoring of the construct presence, and, through engineering of excitation properties, could allow for alternate colours and through Forster Resonance Energy Transfer (FRET) may be used to optically measure the extent of polymerisation through the carrying of optical energy due to quantum tunnelling as polymerisation brings copies of the GFP on separate copies of the construct into close proximity. In each case a different Camelid would be incorporated specific to that antigen.

Typically, the binding affinity for a given target of Camelids and other such scaffold type programmable affinity proteins would be reversibly disrupted by bringing the environmental condition from a neutral pH to an acidic one as in Examples 1 to 3. Additionally, such bonding is also reversibly disrupted through the addition of moderate concentrations of denaturant such as 1-3M of Guanidinium Hydrochloride or Urea, or some non-aqueous solvents such as methanol, ethanol or ethylene glycol. Thus, such a construct could bind to copies of itself forming polymer as in the prior examples, reversibly monomerise through changes in the buffering environment, and bind and copurify copies of the partner antigen from an impure mixture in the same way as Examples 1-3 were able to purify IgG antibody.

The programmability of the Camelid antibody fragment means that it could similarly replicate the function of the Protein A and Protein L in the prior examples in being raised to specifically bind IgG antibodies or antibody fragments with the Antibody Fc domain or Kappa Light chain as the partner ligand incorporated in the construct and full length antibody being co-purified from a mixture as in the prior examples. Furthermore as multiple copies of Camelid antibody fragments are incorporated into the construct, different Camelids and different quantities of each may be incorporated, allowing a primary set of the incorporated Camelid(s) to bind to an arbitrary protein ligand partner incorporated into the construct to provide the polymerisation function such as the mentioned proteins, and a second set of the incorporated Camelid(s) to bind to an arbitrary target ligand that is not part of the construct and is to be purified. Increasing quantities of the first type in the construct would increase the rate and level of polymerisation and polymer branching, and increasing quantities of the second type would increase affinity for the target ligand to be purified. Moreover, having both as distinct groups would allow further differentiation in binding propensity in the environmental pH allowing better selective unbinding—thus as an example:

The environment pH could be lowered such that a Camelid incorporated on the construct and raised to bind antibodies would release the antibodies, while, in the same condition, a second Camelid incorporated on the same construct and raised to bind GFP that was itself incorporated into the construct might remain bound and the construct conglomerate would remain polymerised and thus may be sifted from the solution leaving the antibody behind.

Camelid fragment proteins are one class of antibody or antibody mimetic. Consequently, any similar type of system may be used in its place in a similar way, including, but not limited to engineered single chain variants of human antibodies and antibody fragments, Affibodies (derived from Protein A Z-domains), Monobodies (fibronection derived), DARPins (Ankyrin derived), Affimers (Cystatin derived), Knob domain peptides (Bovine antibody derived) and more. In all cases the pI of the construct and the pI of the construct polymer and the pI of the construct/target protein polymer complex may be modulated and tuned by the inclusion of the charge component into the construct. By controlling the quantity and ratio of histidine to alternative charged amino acids in the charge component incorporated at the tail and in the linkers, the overall pI can be shifted to both modulate the polymerisation/monomerisation conditions into a favourable range, and to control the electrostatic charge while polymerising by setting the pH to either side of the programmed pI but within the polymerisation range, thus giving the polymer positive, negative, or neutral overall charge to selectively repel positive, negative charges and modulate hydrophobic sequestration respectively.

### Example 8

Another proposed construct example system would include reversibly binding DNA aptamers as an example of oligonucleotide with programmable binding ability. Similar to human antibodies these Aptamers can be raised through library selection against a suitable protein target based antigen to achieve very high binding affinity and specificity. They have the added benefit of being compact and composed of nucleotides alone, making them industrially manufacturable by chemical solid synthesis means amongst others, and conferring safe biocompatibility with minimal immune response and high purifyability via conventional means. They may be further engineered or further selected to take advantage of tertiary structures to tune target affinity and confer reversible binding depending on environmental conditions i.e. an environmental change could induce a conformational shift that significantly reduces their binding affinity for the target. Thus such a programable system may be used to rapidly create a class of programmable affinity. In this example the Aptamer may be incorporated as the (Lock) affinity binding partner in the construct, with its specific antigen partner (Key) being the target of interest. Furthermore by the nature of Oligonucleotide binding, an incorporated oligonucleotide sequence may be engineered to be complimentary to another incorporated oligonucleotide sequence and strategically placed into the construct, or even multiple copies in unmatching ratios, and can allow two copies of the construct to bind to each other, forming polymers. One of these sequences may be a part of the aptamer section itself. Self-binding within one copy of the construct could be limited both through the strategic placement of complimentary sequence: for example, having each of the complementary sequences separated by a more stable and large tertiary structure prevents them reaching each other and therefore conformationally blocks self binding but allows multiple construct copies to polymerise together.

Typically the binding affinity for a given target of an Aptamer and other such programmable affinity oligonucleotides would be reversibly disrupted by bringing the environmental condition from a baseline or operating temperature to a higher one. However, such Aptamers can also be iteratively selected so such bonding is also reversibly disrupted through changes in pH or the addition of non aquespous solvents such as methanol, ethanol or ethylene glycol. Thus, such a construct incorporating such aptamers could bind to copies of itself forming polymer as in the prior examples, reversibly monomerise through changes in the solution environment, and bind and copurify copies of the target from an impure mixture in the same way as examples 1-3 were able to purify IgG antibody.

Multiple copies of the Aptamer could be incorporated into the construct and increasing quantities of Aptamers in the construct would increase the affinity and binding ratio for the target ligand to be purified. Similarly, the construct may incorporate multiple copies of the complimentary sequences and would affect the rate and level of polymerisation and polymer branching.

Such an Aptamer based construct could be constructed incorporating the charge component, which may be achieved in more than one way. One such way is to incorporate an additional Aptamer that is selected to specifically bind a polypeptide that contains the charge component as per the other examples, thus conferring a group charge and programmable pI as in the other cases. Another way to achieve a similar effect is to incorporate modified bases through solid phase chemical synthesis means that may directly incorporate peptide or amino acid groups, or may alternatively contain charge carrying reactive groups directly, with a similar assortment of pKa as to their amino-acid counterparts allowing programmable pIs in the same manner as the provided polypeptide based examples.

The option of incorporating non-standard oligonucleotide bases through artificial chemical synthesis means also opens the possibility for alternative chemistries with complex and customisable groups to augment conformation of aptamer tertiary structures, or to modulate the binding of an aptamer complex to the purification target, potentially to enhance the binding, or affect it in such a way as to tune the equilibrium and reversibility of the binding.

In a similar way to the provided Camelid example, tuning of the Aptamer's binding affinity to the purification target in response to the environmental condition is advantageous in controlling its affinity relative to the complimentary binding of the construct polymerising region; by creating a condition where the latter may bind in a particular environment while the former does not would allow the constructs to polymerise into a gel state while leaving the target behind, as a final self-purification step to remove the construct from the target.

1. A composition comprising a first construct, wherein the first construct comprises:
a charge component;
a lock component; and
a key component;
wherein the lock component and the key component are specific affinity binding partners and are separated such that they are unable to form a binding pair within the same construct;
whereby in a first environmental condition, the charge component imparts a first overall charge to the first construct, the lock component binds to its binding partner on a second construct, and the key component binds to its binding partner on a third construct; and
whereby in a second environmental condition, the charge component imparts a second overall charge to the first construct, and the lock and key components are in an unbound state.

2. The composition of claim 1, further comprising a target compound free in solution, and wherein the target compound is a specific affinity binding partner to the key component.

3. The composition of claim 2, whereby in a third environmental condition, the charge component imparts a third overall charge to the first construct, the lock component binds to its binding partner on a second construct, and the key component binds to its binding partner on a third construct, and the target compound remains unbound.

4. The composition of claim 1, wherein the first construct further comprises a second key component.

5. The composition of claim 4, wherein the lock component and the second key component are specific affinity binding partners, and wherein the lock component and 50 the second key component are separated such that they are unable to form a binding pair within the same construct.

6. The composition of claim 1, wherein the first construct further comprises a second lock component.

7. The composition of claim 6, wherein the second lock component and the key component are specific affinity binding partners, and wherein the second lock component and the key component are separated such that they are unable to form a binding pair within the same construct, optionally wherein the second lock component is a modified version of the first lock component.

8. The composition of claim 1, wherein the composition further comprises a target construct comprising a target compound, and the first construct further comprises a second key component, wherein the target compound is a specific affinity binding partner to the first key component and the second key component.

9. The composition of claim 1, wherein the lock component and the key component of the first construct are oriented such that in the first environmental condition, the lock component binds to its binding partner on a second construct and the key component cannot bind to its binding partner on the second construct.

10. The composition of claim 1, wherein the first construct is a polypeptide.

11. The composition of claim 1, wherein the first environmental condition is a first pH, and the second environmental condition is a second pH, optionally wherein the first pH is between 5 to 9 and the second pH is between 2 to 6, and optionally wherein the second pH is at least 1.5 pH units lower than the first pH.

12. The composition of claim 1, wherein:
(i) the key component is an antigen and the lock component is an antibody specific to the antigen, optionally wherein the antibody is a monoclonal antibody, a fragment antibody, a Bi-specific T-cell engager, an affibody or a Cam elid;
(ii) the key component is a surface cell wall protein (such as Protein A, Protein L, or Protein G) and the surface cell wall protein specifically binds an antibody which is a lock component, optionally wherein the antibody is a monoclonal antibody, a fragment antibody, a Bi- specific T-cell engager, an affibody or a Cam elid;
(iii) the key component is a hormone and the lock component is a hormone receptor specific for the hormone;
(iv) the key component is a signal transduction pathway protein and the lock component is a receptor specific for the signal transduction pathway protein;
(v) the key component is an interfering RNA and the lock component is an oligonucleotide target;
(vi) the key component is a DNA repressor protein and the lock component is a counterpart oligonucleotide; or
(v) the key component is a DNA aptamer and the lock component is a protein ligand.

13. The composition of claim 1, wherein:
(i) the lock component is an antibody, an antibody fragment or an antibody mimetic; and/or,
(ii) the key component is selected from a surface cell wall protein A, a surface cell wall protein L and a surface cell wall protein G.

14. The composition of claim 1, wherein the charge component is a polypeptide comprising between 2 and 8 histidine residues and between 2 and 8 aspartate and/or glutamate residues.

15. The composition of claim 14, wherein:
(i) the histidine residues are clustered in a first group and the aspartate and/or glutamate residues are clustered in a second group; or
(ii) the histidine residues are interspersed with the aspartate and/or glutamate residues.

16. The composition of claim 1, wherein the first construct is linear and the components are separated by rigid interlinks between neighbouring components.

17. The composition of claim 1, wherein the second construct is identical to the first construct; and/or the third construct is identical to the first construct.

18. A method of purification of a target compound, comprising the steps:
i) incubating a composition according to claim 1 and a sample comprising a target compound at a first environmental condition, wherein the first construct binds to the target compound, and the first construct and the second construct bind together and polymerise to form a polymer;
ii) separating the polymer, wherein the target compound remains bound to the polymer;
iii) incubating the polymer at a second environmental condition, wherein the target compound dissociates from the polymer, and the first construct and the second construct dissociate and monomerise;
iv) repeating steps i to iii for between 1 to 200 cycles;
v) separating the target compound from the first construct and the second construct.

19. The method of claim 18, wherein the separation step is a coarse separation such as sifting, filtration or centrifugation.

20. The method of claim 18, wherein the first environmental condition is a first pH, and the second environmental condition in a second pH, optionally wherein the first pH is between 5 to 9 and the second pH is between 2 to 6, and optionally wherein the second pH is at least 1.5 pH units lower than the first pH.

21. A nucleic acid sequence encoding the first construct of the composition according to claim 1.

22. A nucleic acid sequence according to SEQ ID NO: 1.

23. A vector comprising the nucleic acid sequence of claim 22.

24. A host cell comprising the composition of any claim 1.

25. A polypeptide comprising an amino acid sequence according to SEQ ID NO: 2 or SEQ ID NO: 6.


SEQUENCE LISTING (7 sequences)

SEQ ID NO: 1 - DNA, 1416 bp, Artificial Sequence
  Example 5 Construct DNA sequence
ttcaacaaagaacagcagaacgccttttatgaaattctgcatctgccgaatctgaatgaagaacagcgtaatggttttatccagagcctgaaagatgatccgagccagagcgcaaatctgctggcagaagcagcagaagccgcagcaaaagcaccgagcgtttttctgtttccgcctaaaccgaaagataccctgatgattagccgtacaccggaagttacctgtgttgttgttgatgttagccatgaagatccgcaggttaaattcaattggtatgttgatggtgttcaggtgcataacgcaaaaaccaaaccgcgtgaacagcagtataattcaacctatcgtgttgttagcgttctgaccgttctgcatcagaattggctggatggtaaagaatacaaatgcaaggttagcaataaagcactgcctgcaccgattgaaaaaaccatttcaaaagcaaaaggtcagcctcgtgaaccgcaggtttataccctgcctccgagccgtgaagaaatgaccaaaaatcaggttagcctgacctgtctggtgaaaggtttttatccgagcgatattgcagttgaatgggaaagcaatggtcagccggaaaataactataaaaccacaccgcctgttctggatagtgatggtagcttttttctgtatagcaaactgaccgttgataaaagccgttggcagcagggtaatgtttttagctgtagcgtgatgcatgaagccctgcataatcattatacccagaaaagcctgagcctgagcgcagaggcagctgcgaaagcaccgtcagtgtttctgttccctccgaaacctaaggatacgttaatgatttcacgtacccctgaagtgacctgcgtggtggtggatgtttctcatgaggatccacaggtgaagtttaactggtatgtggacggcgtgcaggttcacaatgccaaaacaaaacctcgcgagcagcaatacaatagcacctatcgcgtggtttcagtgctgacagtgctgcaccagaactggttagacggcaaagaatataagtgtaaagtgagcaacaaagcattaccggctcctatcgaaaaaacaatcagcaaagccaaaggccagccacgcgagcctcaagtgtatacgctgccaccgtcacgcgaagagatgacaaaaaaccaagtttcactgacgtgcctggttaaaggcttctatccttcagatatcgccgtggaatgggaatcaaacggccagcctgaaaacaattacaaaacaacccctccggttctggattcagatggttcatttttcctgtactcaaaactgacagtggacaaatcacgctggcagcaaggcaacgttttttcatgttcagttatgcacgaagcactgcacaaccactatacacaaaaatcactgagcctgtcc

SEQ ID NO: 2 - protein, 472 aa, Artificial Sequence
  Example 5 construct polypeptide sequence
FNKEQQNAFYEILHLPNLNEEQRNGFIQSLKDDPSQSANLLAEAAEAAAKAPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSAEAAAKAPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLS

SEQ ID NO: 3 - protein, 44 aa, Artificial Sequence
  B-domain
FNKEQQNAFYEILHLPNLNEEQRNGFIQSLKDDPSQSANLLAEA

SEQ ID NO: 4 - protein, 7 aa, Artificial Sequence
  Linker
AEAAAKA

SEQ ID NO: 5 - protein, 207 aa, Artificial Sequence
  Fc domain
PSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLS

SEQ ID NO: 6 - protein, 362 aa, Artificial Sequence
  Example 2 construct polypeptide sequence
VTIKVNLIFADGKTQTAEFKGTFEEATAEAYRYADLLAKVNGEYTADLEDGGYTINIKFAGKPKPKPKPVTIKENIYFEDGTVQTATFKGTFAEATAEAYRYADLLSKEHGKYTADLEDGGYTINIRFAGKPKPKPKPELVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTVAAPSVFIFPPSDEQLKSGTASVVCLLNNFYPREAKVQWKVDNALQSGNSQESVTEQDSRDSTYSLGSTLTLSKADYEKHKVYACEVTHQGLSSPVTKSFNRGECHHHHKHHHH

SEQ ID NO: 7 - protein, 351 aa, Artificial Sequence
  Example 3 construct polypeptide sequence
VTIKVNLIFADGKTQTAEFKGTFEEATAEAYRYADLLAKVNGEYTADLEDGGYTINIKFAGAEAAAKAVTIKENIYFEDGTVQTATFKGTFAEATAEAYRYADLLSKEHGKYTADLEDGGYTINIRFAGAEAAAKAELVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTVAAPSVFIFPPSDEQLKSGTASVVCLLNNFYPREAKVQWKVDNALQSGNSQESVTEQDSRDSTYSLGSTLTLSKADYEKHKVYACEVTHQGLSSPVTKSFNRGEC